(19) United States
(12) Patent Application Publication
(10) Pub. No.: US 2026/0120287 A1
(43) Pub. Date: Apr. 30, 2026

(54) IMAGE PROCESSING DEVICE, IMAGE PROCESSING METHOD, AND STORAGE MEDIUM

(71) Applicant: NEC Corporation, Tokyo (JP)

(72) Inventor: Masahiro SAIKOU, Tokyo (JP)

(73) Assignee: NEC Corporation, Tokyo (JP)

(21) Appl. No.: 19/419,208

(22) Filed: Dec. 15, 2025

Related U.S. Application Data
(63) Continuation of application No. 18/574,126.

(30) Foreign Application Priority Data
Feb. 27, 2023 (WO) PCT/JP2023/007007

(51) Int. Cl. G06T 7/00; A61B 1/00; A61B 5/00; G06T 3/40; G06T 7/50; G06T 11/00; G16H 30/20; G16H 30/40; G16H 50/20

(52) U.S. Cl. CPC G06T 7/0016; A61B 1/000094; A61B 1/000096; A61B 1/00045; A61B 5/4842; G06T 3/40; G06T 7/0012; G06T 7/50; G06T 11/00; G16H 30/20; G16H 30/40; G16H 50/20; G06T 2207/10068; G06T 2207/20076; G06T 2207/20081; G06T 2207/20084; G06T 2207/30096; G06T 2207/30168

(57) ABSTRACT

The image processing device 1X includes an acquisition means 30X, a detection means 31X, a first determination means 32X, and a second determination means 33X. The acquisition means 30X acquires an endoscopic image of an examination target. The detection means 31X detects, based on the endoscopic image, a lesion region which is a candidate lesion region of the examination target in the endoscopic image. The first determination means 32X determines whether or not the endoscopic image is an image adequate to determine a degree of progression or an invasion depth, based on a size of the lesion region and/or a degree of reliability regarding a probability of the lesion region as the lesion. The second determination means 33X determines the degree of progression or the invasion depth, based on the endoscopic image determined to be the image adequate to determine the degree of progression or the invasion depth.

This application is a Continuation of U.S. application Ser. No. 18/574,126 filed on Dec. 26, 2023, which is a National Stage Entry of PCT/JP2023/031840 filed on Aug. 31, 2023, which claims priority from PCT International Application PCT/JP2023/007007 filed on Feb. 27, 2023, the contents of all of which are incorporated herein by reference, in their entirety.

### TECHNICAL FIELD

The present disclosure relates to a technical field of an image processing device, an image processing method, and a storage medium for processing an image to be acquired in endoscopic examination.

### BACKGROUND

There is known an image processing system which processes a photographed image of a lumen of an organ. For example, Patent Literature 1 discloses a medical image processing device which detects a lesion candidate from a medical image and identifies the degree of malignancy of the detected lesion candidate and the organ included in the medical image.

### CITATION LIST

### Patent Literature

Patent Literature 1: JP 2021-083821A

### SUMMARY

### Problem to be Solved

When the degree of progression (including the invasion depth, hereinafter the same) is determined from an endoscopic image, endoscopic images obtained in time series include many noisy images such as a blurring image, a shinning image, and a splashing image, which are not adequate to determine the degree of progression. In addition, even such an image in which a possible lesion part is photographed with little noise does not always become an image adequate to determine the degree of progression.

In view of the above-described issue, it is therefore an example object of the present disclosure to provide an image processing device, an image processing method, and a storage medium capable of suitably determining the degree of progression.

### Means for Solving the Problem

One mode of the image processing device is an image processing device including:
an acquisition means configured to acquire an endoscopic image obtained by photographing an examination target;
a detection means configured to detect, based on the endoscopic image, a lesion region which is a candidate region of a lesion of the examination target in the endoscopic image;
a first determination means configured to determine whether or not the endoscopic image is an image adequate to determine a degree of progression or an invasion depth, based on at least one of a size of the lesion region and/or a degree of reliability regarding a probability of the lesion region as the lesion; and
a second determination means configured to determine the degree of progression or the invasion depth, based on the endoscopic image determined to be the image adequate to determine the degree of progression or the invasion depth.

One mode of the image processing method is an image processing method executed by a computer, the image processing method including:
acquiring an endoscopic image obtained by photographing an examination target;
detecting, based on the endoscopic image, a lesion region which is a candidate region of a lesion of the examination target in the endoscopic image;
determining whether or not the endoscopic image is an image adequate to determine a degree of progression or an invasion depth, based on at least one of a size of the lesion region and/or a degree of reliability regarding a probability of the lesion region as the lesion; and
determining the degree of progression or the invasion depth, based on the endoscopic image determined to be the image adequate to determine the degree of progression or the invasion depth.

One mode of the storage medium is a storage medium storing a program executed by a computer, the program causing the computer to:
acquire an endoscopic image obtained by photographing an examination target;
detect, based on the endoscopic image, a lesion region which is a candidate region of a lesion of the examination target in the endoscopic image;
determine whether or not the endoscopic image is an image adequate to determine a degree of progression or an invasion depth, based on at least one of a size of the lesion region and/or a degree of reliability regarding a probability of the lesion region as the lesion; and
determine the degree of progression or the invasion depth, based on the endoscopic image determined to be the image adequate to determine the degree of progression or the invasion depth.

### Effect

An example advantage according to the present invention is to suitably determine the degree of progression (including invasion depth).

### BRIEF DESCRIPTION OF THE DRAWINGS

FIG. 1 It illustrates a schematic configuration of the endoscopic examination system.

FIG. 2 It illustrates a hardware configuration of the image processing device.

FIG. 3 It is a diagram showing an outline of a progression determination process performed by the image processing device according to the first example embodiment.

FIG. 4 It illustrates an example of a function blocks of the progression determination process in the first example embodiment.

FIG. 5 It illustrates a first display example displayed by the display device in the endoscopic examination.

FIG. 6 It illustrates a second display example displayed by the display device in the endoscopic examination.

FIG. 7 It is an example of a flowchart showing an outline of a process performed by the image processing device during the endoscopic examination in the first example embodiment.

FIG. 8 It is a schematic configuration diagram of an endoscopic examination system in a modification.

FIG. 9 It is a block diagram of an image processing device according to a second example embodiment.

FIG. 10 It illustrates an example of a flowchart executed by the image processing device in the second example embodiment.

### EXAMPLE EMBODIMENTS

Hereinafter, example embodiments of an image processing device, an image processing method, and a storage medium will be described with reference to the drawings.

### First Example Embodiment

### (1) System Configuration

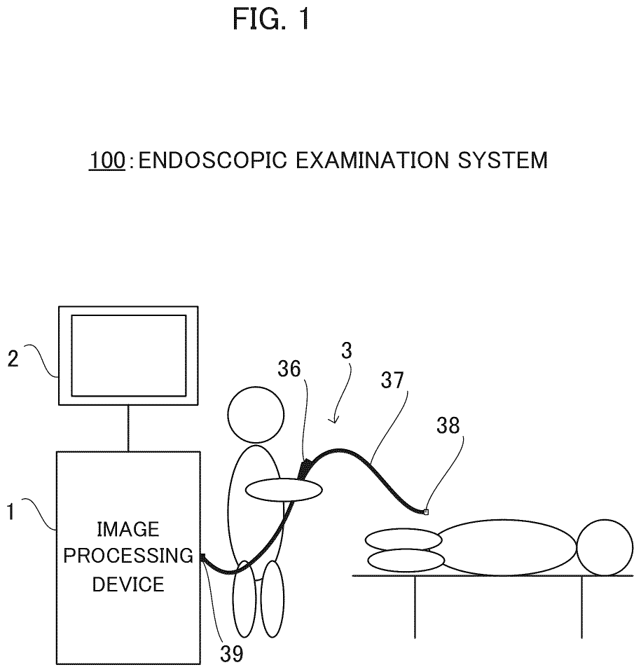

FIG. 1 shows a schematic configuration of an endoscopic examination system 100. As shown in FIG. 1, the endoscopic examination system 100 is a system that detects a lesion part that is a part of an examination target suspected of a lesion based on an image captured by an endoscope to thereby determine the degree of progression of the detected lesion part and present the determination result thereof. This allows the endoscopic examination system 100 to assist an examiner such as a doctor to perform decision making, such as determination of the way to operate the endoscope, and determination of a treatment policy for the subject of the examination. Hereafter, the “degree of progression” may refer to the degree (grade) of comprehensive lesion progression in which the invasion depth (degree of invasiveness) is considered, or may refer to the invasion depth (degree of invasiveness) itself. The endoscopic examination system 100 mainly includes an image processing device 1, a display device 2, and an endoscope 3 connected to the image processing device 1 and manipulated by an examiner such as a doctor who conducts examination or treatment.

The image processing device 1 acquires an image (also referred to as “endoscopic image Ia”) captured by the endoscope 3 in time series from the endoscope 3 and displays a screen image based on the endoscopic image Ia on the display device 2. The endoscopic image Ia is an image captured at a predetermined frame rate in at least one of the insertion process of the endoscope 3 to the subject and/or the ejection process of the endoscope 3 from the subject. In the present example embodiment, the image processing device 1 detects, from endoscopic images Ia in time series, each endoscopic image Ia in which a candidate region (also referred to as “lesion regions”) of a lesion part is included. Then, the image processing device 1 selects an image adequate to determine the degree of progression of the lesion from the detected endoscopic images Ia to make a determination of the degree of progression of the lesion in the selected image and present the information relating to the determination result. In addition, the image processing device 1 outputs a suggestion regarding photography to acquire an image adequate to determine the degree of progression of the lesion when such an image cannot be acquired.

The display device 2 is a display or the like for display information based on the display signal supplied from the image processing device 1.

The endoscope 3 mainly includes an operation unit 36 for examiner to perform a predetermined input, a shaft 37 which has flexibility and which is inserted into the organ to be photographed of the subject, a tip unit 38 having a built-in photographing unit such as an ultra-small image pickup device, and a connecting unit 39 for connecting with the image processing device 1.

The configuration of the endoscopic examination system 100 shown in FIG. 1 is an example, and various change may be applied thereto. For example, the image processing device 1 may be configured integrally with the display device 2. In another example, the image processing device 1 may be configured by a plurality of devices.

It is noted that the target of the endoscopic examination in the present disclosure may be any organ subject to endoscopic examination such as large bowel, esophagus, stomach, and pancreas. Examples of the target of the endoscopic examination in the present disclosure include a laryngendoscope, a bronchoscope, an upper digestive tube endoscope, a duodenum endoscope, a small bowel endoscope, a large bowel endoscope, a capsule endoscope, a thoracoscope, a laparoscope, a cystoscope, a cholangioscope, an arthroscope, a spinal endoscope, a blood vessel endoscope, and an epidural endoscope. A disorder (condition) of the lesion part subjected to detection in the endoscopic examination are exemplified as (a) to (f) below.
(a) Head and neck: pharyngeal cancer, malignant lymphoma, papilloma
(b) Esophagus: esophageal cancer, esophagitis, esophageal hiatal hernia, Barrett's esophagus, esophageal varices, esophageal achalasia, esophageal submucosal tumor, esophageal benign tumor
(c) Stomach: gastric cancer, gastritis, gastric ulcer, gastric polyp, gastric tumor
(d) Duodenum: duodenal cancer, duodenal ulcer, duodenitis, duodenal tumor, duodenal lymphoma
(e) Small bowel: small bowel cancer, small bowel neoplastic disease, small bowel inflammatory disease, small bowel vascular disease
(f) Large bowel: colorectal cancer, colorectal neoplastic disease, colorectal inflammatory disease; colorectal polyps, colorectal polyposis, Crohn's disease, colitis, intestinal tuberculosis, hemorrhoids.

### (2) Hardware Configuration

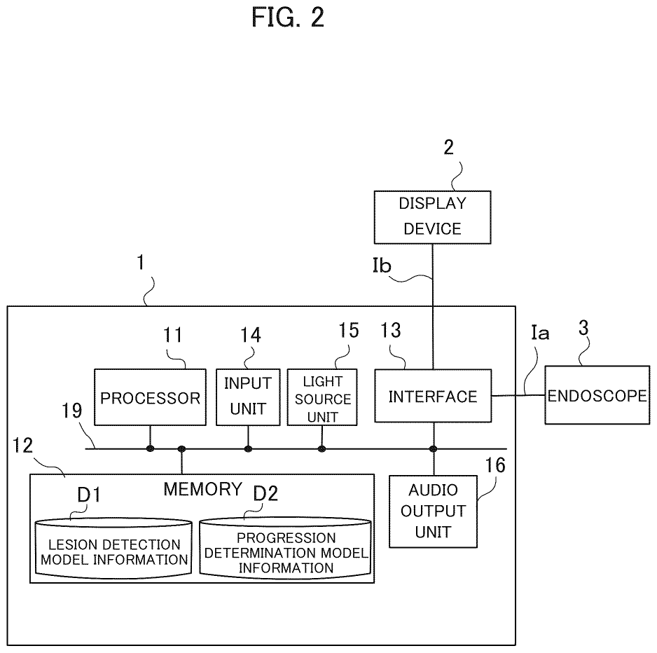

FIG. 2 shows a hardware configuration of the image processing device 1. The image processing device 1 mainly includes a processor 11, a memory 12, an interface 13, an input unit 14, a light source unit 15, and an audio output unit 16. Each of these elements is connected to one another via a data bus 19.

The processor 11 executes a predetermined process by executing a program or the like stored in the memory 12. The processor 11 is one or more processors such as a CPU (Central Processing Unit), a GPU (Graphics Processing Unit), and a TPU (Tensor Processing Unit). The processor 11 may be configured by a plurality of processors. The processor 11 is an example of a computer.

The memory 12 is configured by a variety of volatile memories which is used as working memories, and nonvolatile memories which stores information necessary for the process to be executed by the image processing device 1, such as a RAM (Random Access Memory) and a ROM (Read Only Memory). The memory 12 may include an external storage device such as a hard disk connected to or built in to the image processing device 1, or may include a storage medium such as a removable flash memory. The memory 12 stores a program for the image processing device 1 to execute each process in the present example embodiment.

Further, the memory 12 stores lesion detection model information D1 which is information regarding a lesion detection model and progression determination model information D2 which is information regarding a progression determination model. Details of the lesion detection model and the progression determination model will be described later. Further, the memory 12 may optionally include other information necessary for the image processing device 1 to perform each process in the present example embodiment.

The interface 13 performs an interface operation between the image processing device 1 and an external device. For example, the interface 13 supplies the display information “Ib” generated by the processor 11 to the display device 2. Further, the interface 13 supplies the light generated by the light source unit 15 to the endoscope 3. The interface 13 also provides an electrical signal to the processor 11 indicative of the endoscopic image Ia supplied from the endoscope 3. The interface 13 may be a communication interface, such as a network adapter, for wired or wireless communication with the external device, or a hardware interface compliant with a USB (Universal Serial Bus), a SATA (Serial AT Attachment), or the like.

The input unit 14 generates an input signal based on the operation by the examiner. Examples of the input unit 14 include a button, a touch panel, a remote controller, and a voice input device. The light source unit 15 generates light for supplying to the tip unit 38 of the endoscope 3. The light source unit 15 may also incorporate a pump or the like for delivering water and air to be supplied to the endoscope 3. The audio output unit 16 outputs a sound under the control of the processor 11.

Next, the details of the lesion detection model and the progression determination model will be described.

The lesion detection model is a machine learning model configured to generate an inference result regarding a lesion region falling under a target disorder of detection in the endoscopic examination, and parameters required for the model are stored in the lesion detection model information D1. For example, the lesion detection model is configured to output, when an endoscopic image is inputted thereto, an inference result regarding a lesion region in the inputted endoscopic image. In other words, the lesion detection model is a model which learned a relation between an input image to the lesion detection model and the lesion region in the input image. The lesion detection model may be any model (including statistical models, and the same applies hereinafter) equipped with an architecture used in any machine learning, such as a neural network and a support vector machine. Examples of the typical models of such a neural network include Fully Convolutional Network, SegNet, U-Net, V-Net, Feature Pyramid Network, Mask R-CNN, and DeepLab. When the lesion detection model is constituted by a neural network, the lesion detection model information D1 includes various parameters regarding, for example, a layer structure, a neuron structure of each layer, the number of filters and filter sizes in each layer, and a weight for each element of each filter.

Specific modes (first output mode and second output mode) of the inference result outputted by the lesion detection model will be described.

According to the first output mode, the lesion detection model is configured to output, as an inference result, a map indicating the degree (also referred to as “lesion reliability degree”) of reliability of the presence of the lesion region for each unit region in the inputted endoscopic image. The above-described map is also referred to as “lesion reliability map” hereinafter. For example, the lesion reliability map is an image showing lesion reliability degrees for respective pixels (which may be subpixels) or for respective pixel blocks defined according to a predetermined rule. It is herein assumed that the higher value the lesion reliability degree of a region indicates, the higher the degree of reliability of the region as a lesion region becomes. The lesion reliability map may be a mask image indicating the lesion region by binary.

In the second output mode, the lesion detection model is configured to output an inference result indicating, respectively, a bounding box indicating the existence range of a lesion region in the inputted endoscopic image and the degree of reliability of the region surrounded by the bounding box as a lesion region. The degree of reliability herein indicates, for example, a confidence score indicating the degree of confidence outputted from the output layer of a neural network when the lesion detection model is constituted by a neural network. The above-described modes of the inference result outputted by the lesion detection model are examples, and an inference result according to any mode may be outputted from the lesion detection model.

The lesion detection model is trained in advance based on sets of an input image that conforms to the input format of the lesion detection model and correct answer data (in the above-described example, a correct lesion reliability map or a correct bounding box) that indicates the correct answer of the inference result that the lesion detection model should output when the input image is inputted thereto. Then, parameters of the models obtained through the training are stored in the memory 12 as the lesion detection model information D1.

It is noted that the lesion detection model may include a feature extraction model for extracting features from an endoscope image or may be a model separate from the feature extraction model. In the latter case, the lesion detection model is a model trained to output the inference result described above when features (a tensor with a predetermined number of dimensions) outputted by the feature extraction model in response to the input of an endoscopic image is inputted thereto.

The progression determination model is a machine learning model configured to infer (classify) the degree of progression of a lesion indicated by a lesion region included in an inputted endoscopic image, and the parameters required for the model are recorded in the progression determination model information D2. The progression determination model is configured to output, when an endoscopic image is inputted thereto, an inference result (in detail, a classification result indicating a class of progression) indicating the degree of progression of the lesion in the inputted endoscopic image. In other words, the progression determination model is a model which learned the relation between an input image to the progression determination model and the degree of progression of a lesion in the input image. The progression determination model may be a model (including a statistical model, hereinafter the same) having an architecture used in any machine learning, such as a neural network and a support vector machine. When the progression determination model is configured by a neural network, the progression determination model includes various parameters regarding, for example, a layer structure, a neuron structure of each layer, the number of filters and the size of filters in each layer, and a weight for each element of each filter.

The progression determination model is trained in advance on the basis of sets of an input image that conforms to the input format of the progression determination model and correct answer data (i.e., a class of the degree of progression that is a correct answer) indicating the correct inference result that the progression determination model should output when the input image is inputted thereto. The parameters and the like of the models obtained by learning are stored in the memory 12 as the progression determination model information D2.

It is noted that the endoscopic image inputted to the progression determination model may be a whole image of the endoscopic image Ia generated by the endoscope 3, or may be an image cut out from the endoscopic image (i.e., a partial image of the endoscopic image Ia) so as to include at least the lesion region detected by the lesion detection model. Instead of the endoscopic image, the features of the image calculated by the above-described lesion detection model or feature extraction model may be inputted to the progression determination model.

### (3) Overview

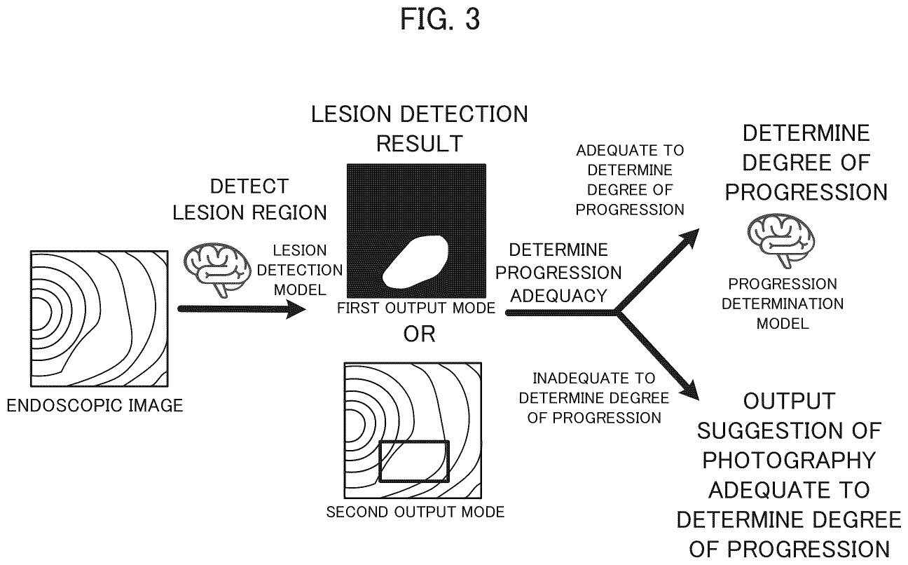

FIG. 3 is a diagram illustrating an outline of a progression determination process relating to determination of the degree of progression made by the image processing device 1 according to the first example embodiment.

First, the image processing device 1 detects a lesion region in each endoscopic image Ia obtained from the endoscope 3 at a predetermined frame rate using the lesion detection model. Then, the image processing device 1 acquires the inference result regarding the lesion region in the endoscopic image Ia from the lesion detection model. In the example shown in FIG. 3, the image processing device 1 acquires, as the lesion detection result, either a lesion reliability map indicating the inference result outputted from the lesion detection model according to the first output mode or a bounding box indicating the inference result outputted from the lesion detection model according to the second output mode. Here, the lesion reliability map is represented in such a state that the higher the lesion reliability degree of a pixel is, the closer to while the color of the pixel becomes. For convenience of explanation, the bounding box described above is superimposed on the endoscopic image Ia inputted to the lesion detection model.

Here, the image processing device 1 does not make a determination of the degree of progression based on the progression determination model for such an endoscopic image Ia in which the lesion region could not be detected. According to the first output mode, the image processing device 1 determines that any unit region (e.g., pixel) whose lesion reliability degree is equal to or larger than a predetermined threshold value (also referred to as “first threshold value”) is a part of a lesion region. Then, upon determining that there is no such a unit region or no connected region of the unit regions constituted by a predetermined number or more of pixels, the image processing device 1 determines that a lesion region cannot be detected. For example, the first threshold value described above is a default value previously stored in the memory 12 or the like. In contrast, according to the second output mode, the image processing device 1 regards the region within the bounding box as a lesion region whereas the image processing device 1 determines that the lesion region could not be detected upon determining that the bounding box cannot be obtained.

Next, the image processing device 1 makes a determination (also referred to as “progression adequacy determination”) of whether or not each endoscopic image Ia in which the lesion region is detected is adequate to determine the degree of progression. In this instance, the image processing device 1 determines the degree of progression regarding the target endoscopic image Ia based on at least one of the size of the lesion region in the target endoscopic image Ia and/or the degree of reliability of the detected lesion region.

Upon determining that the target endoscopic image Ia in which the lesion region is detected is an endoscopic image adequate to determine the degree of progression, the image processing device 1 determines the degree of progression of the lesion region by the progression determination model using the target endoscopic image Ia. Thus, the image processing device 1 can automatically select an endoscopic image Ia adequate to determine the degree of progression of the lesion and determine the degree of progression of the lesion with high accuracy. On the other hand, upon determining that the endoscopic image Ia in which the lesion region is detected is not an endoscopic image adequate to determine the degree of progression, the image processing device 1 outputs a suggestion for photography to acquire the endoscopic image Ia adequate to determine the degree of progression by the endoscope 3 by the display device 2 or the audio output unit 16. Thus, the image processing device 1 can support the examiner to operate the endoscope 3 so as to take an endoscopic image Ia adequate to determine the degree of progression and promote the acquisition of an endoscopic image Ia adequate to determine the degree of progression.

### (4) Functional Blocks

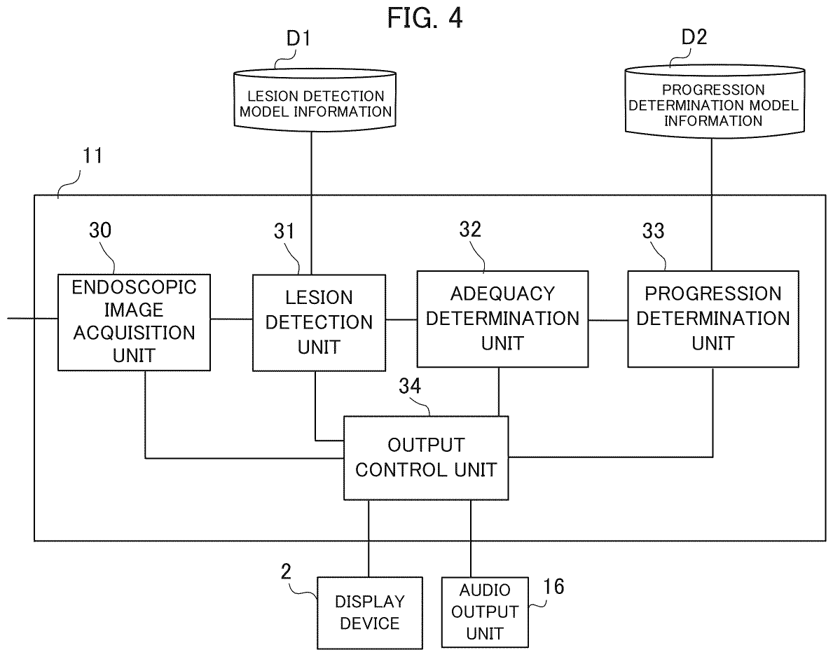

FIG. 4 is an example of the functional blocks of the progression determination process in the first example embodiment. The processor 11 of the image processing device 1 functionally includes an endoscopic image acquisition unit 30, a lesion detection unit 31, an adequacy determination unit 32, a progression determination unit 33, and an output control unit 34. In FIG. 4, any blocks to exchange data with each other are connected to each other by a solid line, but the combination of the blocks to exchange data with each other is not limited thereto. The same applies to the drawings of other functional blocks described below.

The endoscopic image acquisition unit 30 acquires an endoscopic image Ia taken by the endoscope 3 through the interface 13 at predetermined intervals. Then, the endoscopic image acquisition unit 30 supplies the acquired endoscopic image Ia to the lesion detection unit 31 and the output control unit 34, respectively. Then, at the time intervals in which the endoscopic image acquisition unit 30 acquires the endoscopic image Ia, the subsequent processing units perform the processing described later periodically.

The lesion detection unit 31 detects the lesion region in the endoscopic image Ia supplied from the endoscopic image acquisition unit 30, based on the lesion detection model information D1. In this instance, the lesion detection unit 31 inputs the endoscopic image Ia to the lesion detection model configured by referring to the lesion detection model information D1, and acquires the inference result of the lesion detection outputted by the lesion detection model. The inference result of the lesion detection may be a lesion reliability map according to the first output mode and may be a set of the bounding box and the lesion reliability degree according to the second output mode. Upon determining that the lesion region is detected, the lesion detection unit 31 supplies the lesion detection result corresponding to the inference result of the above-described lesion detection to the adequacy determination unit 32 together with the endoscopic image Ia. Further, the lesion detection unit 31 supplies the lesion detection result to the output control unit 34 regardless of whether or not it has detected the lesion region. It is noted that the lesion detection result in the case where the lesion region has not been detected is, for example, information indicating that the lesion region has not been detected.

The adequacy determination unit 32 makes a progression adequacy determination that is a determination of whether or not the endoscopic imaging Ia supplied from the lesion detection unit 31 is adequate to determine the degree of progression of the lesion. In this instance, the adequacy determination unit 32 acquires at least one of the size of the lesion region detected from the endoscopic image Ia and/or the degree of reliability of the detected lesion region on the basis of the inference result of the lesion detection, and makes the progression adequacy determination on the basis of at least one of the acquired size and/or the degree of reliability. Then, upon determining that the endoscopic image Ia is adequate to determine the degree of progression of the lesion, the adequacy determination unit 32 supplies the endoscopic image Ia to the progression determination unit 33. In contrast, upon determining that the endoscopic image Ia is not adequate to determine the degree of progression of the lesion, the adequacy determination unit 32 supplies a determination result (also referred to as “inadequacy determination result”) indicating that the endoscopic image Ia is not adequate to determine the degree of progression of the lesion to the output control unit 34. In some embodiments, even when the endoscopic image Ia is determined to be adequate to determine the degree of progression of the lesion, the adequacy determination unit 32 may supplies a determination result (also referred to as “adequacy determination result”) indicating that the endoscopic image Ia is adequate to determine the degree of progression of the lesion to the output control unit 34.

Based on the progression determination model information D2, the progression determination unit 33 determines the degree of progression (i.e., the class of the progression of the lesion region) of the lesion region included in the endoscopic image Ia that is determined by the progression determination unit 32 to be adequate for use in determining the degree of progression. In this case, for example, the progression determination unit 33 inputs the endoscopic image Ia into the progression determination model configured by referring to the progression determination model information D2 and then acquires the inference result regarding the degree of progression outputted by the progression determination model. In this case, for example, the progression determination model outputs, as the inference result of the degree of progression, the most probable class (e.g., grade or stage representing the degree of progression) of the degree of progression and the confidence score of each candidate class of the degree of progression. Instead of the endoscopic image Ia, the progression determination unit 33 may input a partial image of the endoscopic image Ia including the lesion region or features of the endoscopic image Ia calculated by the lesion detection unit 31 into the progression determination model. Then, the progression determination unit 33 supplies the progression determination result based on the above-described inference result regarding the degree of progression to the output control unit 34. The progression determination result is information obtained through the determination of the degree of progression, and, for example, indicates a class of progression that is determined to be most probable, and the confidence score of the class.

The progression determination unit 33 may determine the degree of progression to be finally outputted to the output control unit 34 based on a predetermined number (two or more) of inference results regarding the degree of progression based on the predetermined number of endoscopic images Ia, instead of determining the degree of progression to be finally outputted to the output control unit 34 based on the inference result regarding the degree of progression based on a single endoscopic image Ia. In this case, if the predetermined number of the inference results regarding the degree of progression outputted by the progression determination model are accumulated, the progression determination unit 33 determines the degree of progression to be outputted by the output control unit 34 based on the predetermined number of accumulated inference results. In this case, for example, the progression determination unit 33 aggregates the predetermined number of the inference results regarding the degree of progression, and determines that the degree of progression as final is the most frequent class (i.e., the class determined by majority voting) among class(es) inferred as the most probable. In this case, for example, the progression determination result outputted by the progression determination unit 33 include the class determined by majority voting, the average value or any other representative value of the confidence scores of the class.

The output control unit 34 generates display information Ib on the basis of the latest endoscopic image Ia supplied from the endoscopic image acquisition unit 30, the lesion detection result outputted by the lesion detection unit 31, and the progression determination result outputted by the progression determination unit 33. Then, the output control unit 34 supplies the generated display information Ib to the display device 2 to thereby cause the display device 2 to display the latest endoscopic image Ia, the lesion detection result, and the progression determination result and the like. In some embodiments, the output control unit 34 may control, based on the lesion detection result, the audio output unit 16 to output a warning sound or voice guidance or the like for notifying the user that the lesion part is detected.

The output control unit 34 outputs, by the display device 2 or the audio output unit 16, a suggestion for photography by the endoscope 3 to acquire an endoscopic image Ia adequate to determine the degree of progression, based on the inadequacy determination result supplied from the adequacy determination unit 32. For example, the output control unit 34 outputs the suggestion described above if inadequacy determination results are consecutively generated for a predetermined number of times or for a predetermined period without generation of an adequacy determination result.

In this example, in the first example of the suggestion, the output control unit 34 outputs the information for prompting the photographing position to approach the lesion region in order to obtain a more enlarged endoscopic image Ia of the lesion region. For example, the output control unit 34 displays or outputs by audio such information “Move the camera closer to the lesion region”.

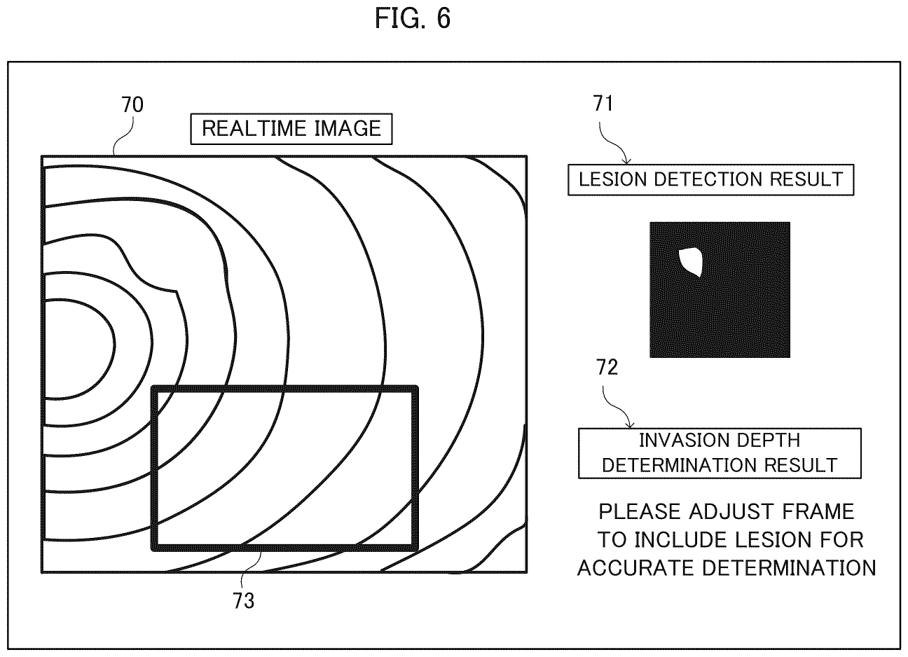

In the second example of the suggestion, the output control unit 34 outputs information indicative of the target range regarding the lesion region on the endoscopic image Ia. For example, the output control unit 34 displays a frame which indicates a preferable display range of the lesion region and which is superimposed on the latest endoscopic image Ia. The display range of the frame superimposed on the endoscope image Ia may be a predetermined range stored in advance in the memory 12 or the like, or may be a range whose shape and/or size is adjusted based on the lesion detection result. Specific aspects of the second example will be described in detail in the display example shown in FIG. 6 to be described later.

In some embodiments, upon determining that, inadequacy determination results are consecutively generated for a predetermined number of times or for a predetermined period without generation of the adequacy determination result even after the output of the suggestion, the output control unit 34 display or output, by audio, information that the degree of progression cannot be determined.

In some embodiments, the output control unit 34 may output the suggestion based on the progression determination result outputted by the progression determination unit 33. for example, if the confidence score of the most probable class of the degree of progression is smaller than a threshold value, the output control unit 34 may output the suggestion according to the above-mentioned first example or second example, regardless of the presence or absence of inadequacy determination result.

In some embodiments, the output control unit 34 may determine and output a coping method (remedy), based on determination result regarding the degree of progression of the examination target and a model generated through machine learning of a correspondence relation between the progression determination result and the coping method. The way to determine the coping method is not limited to the way described above. Outputting a coping method can further assist the examiner's decision making.

Each component of the endoscopic image acquisition unit 30, the lesion detection unit 31, the adequacy determination unit 32, the progression determination unit 33, and the output control unit 34 can be realized, for example, by the processor 11 which executes a program. In addition, the necessary program may be recorded in any non-volatile storage medium and installed as necessary to realize the respective components. In addition, at least a part of these components is not limited to being realized by a software program and may be realized by any combination of hardware, firmware, and software. At least some of these components may also be implemented using user-programmable integrated circuitry, such as FPGA (Field-Programmable Gate Array) and microcontrollers. In this case, the integrated circuit may be used to realize a program for configuring each of the above-described components. Further, at least a part of the components may be configured by a ASSP (Application Specific Standard Produce), ASIC (Application Specific Integrated Circuit) and/or a quantum processor (quantum computer control chip). In this way, each component may be implemented by a variety of hardware. The above is true for other example embodiments to be described later. Further, each of these components may be realized by the collaboration of a plurality of computers, for example, using cloud computing technology.

### (5) Progression Adequacy Determination

Here, a specific description will be given of the progression adequacy determination.

For example, in the case of making the progression adequacy determination based on the size of the lesion region, upon determining that the size of the lesion region is equal to or larger than a predetermined size, the adequacy determination unit 32 generates an adequate determination result. In contrast, upon determining that the size of the lesion region is smaller than the predetermined size, the adequacy determination unit 32 generates an inadequacy determination result. The above-described predetermined size is, for example, a default value determined as a size necessary for accurate determination of the degree of progression, and is stored in advance in the memory 12 or the like. The size of the lesion region in the case of the first output mode is, for example, the size (e.g., the number of pixels) of the connected region of unit regions whose lesion reliability degree is equal to or larger than the first threshold value. In contrast, the size of the lesion region in the case of the second output mode is, for example, the size of the bounding box.

In another example, in the case of making the above-described adequacy determination based on the degree of reliability of the lesion region, upon determining that the degree of reliability of the lesion region is equal to or larger than a predetermined threshold value (also referred to as “second threshold value”), the adequacy determination unit 32 generates an adequacy determination result. In contrast, upon determining that the degree of reliability of the lesion region is less than the second threshold value, the adequacy determination unit 32 generates an inadequacy determination result. The degree of reliability of the lesion region in the case of the first output mode is, for example, the average value, median value, or any other representative value of the lesion reliability degrees of the unit regions constituting the lesion region. In contrast, the degree of reliability of the lesion region in the case of the second output mode is, for example, the degree of reliability associated with the bounding box. The above-described second threshold value is, for example, a default value defined as the degree of reliability required for accurate determination of the degree of progression, and is stored in advance in the memory 12 or the like. In some embodiments, the second threshold value in the case of the first output mode may be set to a value which is equal to or larger than the first threshold value that is compared with the lesion reliability degree for each unit region in determining whether the lesion region is presence or absence.

In yet another example, in the case of making the progression adequacy determination based on both the size and the degree of reliability of the lesion region, upon determining that the size of the lesion region is equal to or larger than the predetermined size and the degree of reliability of the lesion region is equal to or larger than the second threshold value, the adequacy determination unit 32 generates an adequacy determination result. On the other hand, upon determining that the size of the lesion region is less than the predetermined size or the degree of reliability of the lesion region is less than the second threshold value, the adequacy determination unit 32 generates an inadequacy determination result.

In some embodiments, the adequacy determination unit 32 may change the criterion used for the progression adequacy determination based on the progression determination result (that is, information obtained through the determination of the degree of progression) outputted by the progression determination unit 33. The criterion described above corresponds to at least one of the predetermined size to be compared to the size of the lesion region and/or the second threshold value to be compared to the degree of reliability of the lesion region.

For example, on the basis of the confidence score for the most probable class of the degree of progression determined by the degree determination unit 33, the adequacy determination unit 32 changes the criterion used for the progression adequacy determination. In this case, for example, if the confidence score for the most probable class of the degree of progression is smaller than a predetermined threshold value, the adequacy determination unit 32 changes the criterion used for the progression adequacy determination to be a stricter value. In this case, the adequacy determination unit 32 increases the predetermined size or/and the second threshold value used as the criterion by a predetermined value or a predetermined ratio. Thus, the adequacy determination unit 32 tightens the criterion for determining that the endoscopic image Ia is adequate to determine the degree of progression of the lesion, and promotes the determination of the degree of progression by using a strictly selected endoscopic image Ia. The above-described predetermined threshold value, the predetermined value, and the predetermined ratio are, for example, default values, and are stored in advance in the memory 12 or the like. The predetermined size is an example of the “first criterion”, the second threshold value is an example of the “second criterion”.

In another example, based on the degree of change in the most probable class of the degree of progression determined by the degree determination unit 33 in the time series, the adequacy determination unit 32 changes the criterion used for the progression adequacy determination. In this case, for example, if the determined most probable class of the degree of progression is not consistent in a predetermined number of progression determination results obtained in the time series, the adequacy determination unit 32 determines that the progression determination result fluctuates (i.e. not stable) and changes the criterion used for the progression adequacy determination to be a stricter value. In another example, the adequacy determination unit 32 aggreges the most probable class of the degree of progression based on a predetermined number of progression determination results obtained in the time series. Then, if the ratio of the most frequent class is less than a predetermined ratio, the adequacy determination unit 32 changes the criterion used for the progression adequacy determination to be a stricter value.

### (6) Display Examples

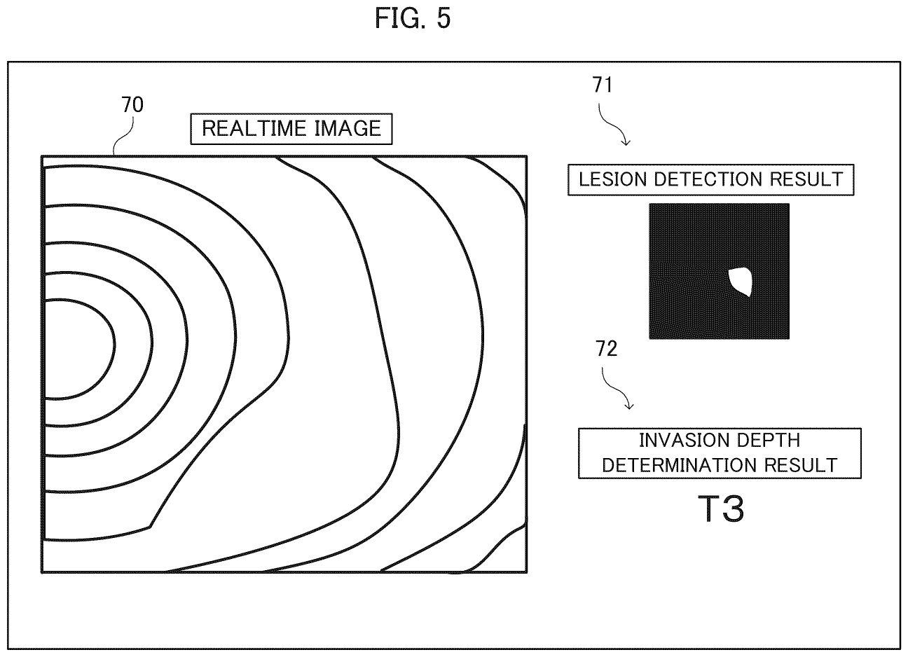

FIG. 5 shows a first display example displayed by the display device 2 in the endoscopic examination. The first display example shows an example of a display screen displayed by the display device 2 when the progression determination unit 33 generates a determination result of the degree of progression (in this case, the invasion depth).

The output control unit 34 of the image processing device 1 outputs the display information Ib generated on the basis of the latest endoscopic image Ia supplied from the endoscopic image acquisition unit 30, the lesion detection result outputted by the lesion detection unit 31, and the progression determination result outputted by the progression determination unit 33 to the display device 2. The output control unit 34 transmits the display information Ib to the display device 2 to thereby display the above-described display screen on the display device 2.

In the first display example shown in FIG. 5, the output control unit 34 of the image processing device 1 provides a real-time image display area 70, a lesion detection result display area 71, and an invasion depth determination result display area 72, on the display screen.

Here, the output control unit 34 displays a moving image representing the latest endoscopic image Ia in the real-time image display area 70. Furthermore, in the lesion detection result display area 71, the output control unit 34 displays the lesion detection result generated by the lesion detection unit 31. Since the lesion detection unit 31 has generated the lesion reliability map as a lesion detection result at the time of displaying the display screen shown in FIG. 5, the output control unit 34 displays an image (here a mask image indicating the lesion region) based on the lesion reliability map on the lesion detection result display area 71. When the lesion detection result indicates a bounding box, the output control unit 34 displays an image obtained by superimposing the above-described bounding box on the latest endoscopic image Ia in the real-time image display area 70 or the lesion detection result display area 71, for example.

In some embodiments, the output control unit 34 may further display a text message to the effect that a lesion is likely to exist in the lesion detection result display area 71, or may output by the audio output unit 16 a sound (including voice) notifying that a lesion is likely to exist.

Furthermore, the output control unit 34 displays, in the invasion depth determination result display area 72, the determination result of the degree of progression (invasion depth) determined by the progression determination unit 33. Here, the progression determination unit 33 determines that the most probable invasion depth belongs to the class “T3”, and the output control unit 34 displays “T3” in the depth determination result display area 72. The output control unit 34 may output the class of the invasion depth determined by the progression determination unit 33 by the audio output unit 16.

According to the first display example, the progression determination unit 33 determines the invasion depth based on endoscopic image Ia adequate to determine the degree of progression of the lesion selected by the adequacy determination unit 32. Therefore, the output control unit 34 can present the determination result regarding the invasion depth with high accuracy to the examiner.

FIG. 6 shows a second display example displayed by the display device 2 in the endoscopic examination. The second display example shows an example of a display screen displayed by the display device 2 when the output control unit 34 suggests the way of image photographing due to the subsequent generation of inadequacy determination results by the adequacy determination unit 32. In the second display example shown in FIG. 6, similarly to the first display example, the output control unit 34 of the image processing device 1 provides a real-time image display area 70, a lesion detection result display area 71, and an invasion depth determination result display area 72 on the display screen. In this example, the output control unit 34 displays, in the lesion detection result display area 71, a mask image based on the lesion detection result in the same way as in the first display example.

In the second display example, in the real-time image display area 70, the output control unit 34 superimposes, on the endoscopic image Ia, a frame 73 representing a preferable target range (i.e., the position and size of the target) of the lesion region in the endoscopic image Ia as a suggestion for photography with the endoscope 3 to acquire the endoscopic image Ia adequate to determine the degree of progression. Further, since it has been unable to determine the invasion depth, the output control unit 34 displays, in the invasion depth determination result display area 72, a message prompting the examiner to adjust the frame 73 to include the lesion region, instead of displaying the determination result of the invasion depth. Accordingly, the examiner who uses the endoscope 3 operates the endoscope 3 so that the outer edge of the lesion region indicated by the lesion detection result display area 71 overlaps with the frame 73. In this case, in some embodiments, the information shown in the lesion detection result display area 71 may be superimposed on the endoscopic image Ia in the real-time image display area 70.

The output control unit 34 may determine at least one of the size and the shape of the frame 73 on the basis of the lesion detection result. For example, the output control unit 34 may increases the size of the frame 73 with an increase in the size of the lesion region detected by the lesion detection unit 31. In another example, the output control unit 34 recognizes the shape of the lesion region detected by the lesion detection unit 31, and determines the shape (e.g., the ratio between the vertical length and horizontal length) of the frame 73 in accordance with the recognized shape. In this case, for example, the output control unit 34 may approximate the lesion region detected by the lesion detection unit 31 with a figure such as an ellipse or a rectangle, and display the frame 73 along the approximated figure.

As described above, according to the second display example, when an endoscope image Ia adequate to determine the degree of progression (here, the invasion depth) cannot be obtained, the image processing device 1 can suggest the way of photography and promote obtaining an endoscope image Ia adequate to determine the degree of progression.

### (7) Processing Flow

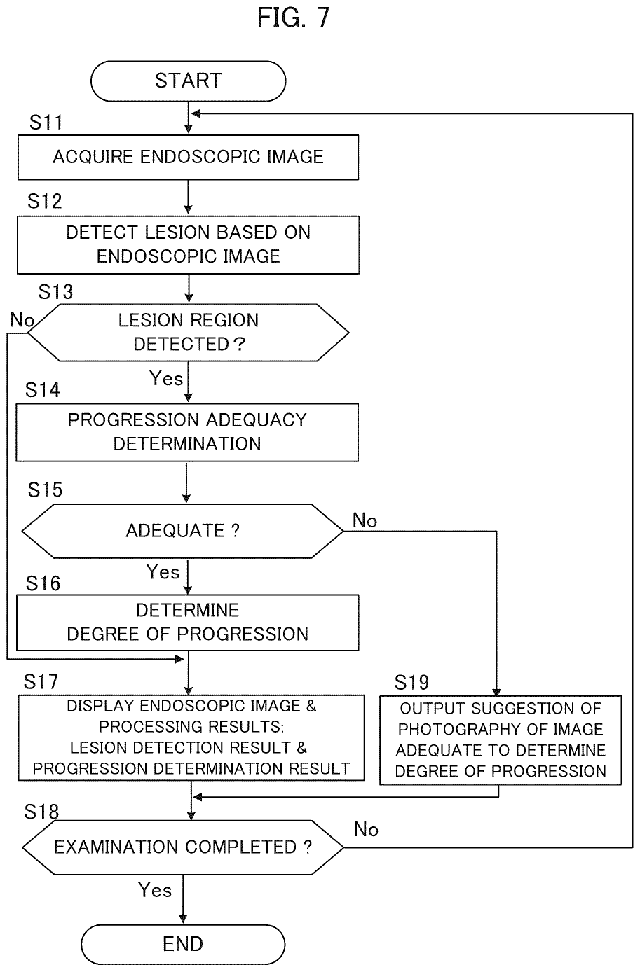

FIG. 7 is an example of a flowchart illustrating an outline of a process that is executed by the image processing device 1 during the endoscopic examination in the first example embodiment.

First, the image processing device 1 acquires the endoscopic image Ia (step S11). In this instance, the endoscopic image acquisition unit 30 of the image processing device 1 receives the endoscopic image Ia from the endoscope 3 through the interface 13.

Next, the image processing device 1 detects a lesion included in the endoscopic image Ia acquired at step S11 (step S12). In this instance, the image processing device 1 acquires the lesion detection result outputted from the lesion detection model by inputting the endoscopic image Ia into the lesion detection model which is built by referring to the lesion detection model information D1.

Then, the image processing device 1 determines whether or not the lesion region is detected from the endoscopic image Ia (step S13). Then, upon determining that the lesion region has been detected from the endoscopic image Ia (step S13; Yes), the image processing device 1 makes a progression adequacy determination for the endoscopic image Ia (step S14). In this case, the image processing device 1 makes the progression adequacy determination on the basis of at least one of the size of the detected lesion region and/or the degree of reliability of the detected lesion region. On the other hand, upon determining that the lesion region has not been detected from the endoscopic image Ia (step S13; No), the image processing device 1 proceeds with the process at step S17 without making the degree of progression determination and the determination of the degree of progression of the lesion.

Then, upon determining that the endoscopic image Ia is adequate to make the progression determination (step S15; Yes), the image processing device 1 determines the degree of progression of the lesion (step S16). In this case, for example, the image processing device 1 acquires the inference result of the degree of progression outputted by the progression determination model when the endoscopic image Ia is inputted to the progression determination model which is built by referring to the progression determination model information D2.

Then, the image processing device 1 displays on the display device 2 information based on: the endoscopic image Ia obtained at step S11; the lesion detection result generated at step S12; and the progression determination result generated at step S16 (step S17). Upon determining that the lesion region has not been detected at step S13, the image processing device 1 displays, at step S1, an endoscopic image Ia and information to the effect that the lesion region has not been detected, for example.

On the other hand, upon determining that the endoscopic image Ia is not adequate to make the progression determination (step S15; No), the image processing device 1 outputs a suggestion regarding the photography to acquire an image adequate to make the progression determination (step S19). The image processing device 1 may execute the process at step S19 only if such a determination at step S15 that the image is inadequate to make the progression determination is consecutively made for a predetermined number of times or for a predetermined period. In this instance, if the number of times or the length of period of such a determination at step S15 that the image is inadequate to make the progression determination does not reach the above-mentioned predetermined number or the length of the above-mentioned predetermined period, the image processing device 1 performs, for example, a process of displaying the endoscopic image Ia and the lesion detection result without outputting the suggestion.

Then, the image processing device 1 determines whether or not the endoscopic examination has been completed after the process at step S17 or step S19 (step S18). For example, the image processing device 1 determines that the endoscopic examination has been completed if a predetermined input or the like to the input unit 14 or the operation unit 36 is detected. Upon determining that the endoscopic examination has been completed (step S18; Yes), the image processing device 1 ends the process of the flowchart. On the other hand, upon determining that the endoscopic examination has not been completed (step S18; No), the image processing device 1 gets back to the process at step S11.

### (8) Modifications

Next, a description will be given of preferred modifications to the first example embodiment described above. The following modifications may be applied to the first example embodiment described above in combination.

### (First Modification)

After the examination, the image processing device 1 may process the video image configured by endoscopic images Ia generated in the endoscopic examination.

For example, when a video image to be processed is designated based on the user input by the input unit 14 at any timing after the examination, the image processing device 1 sequentially performs processing of the flowchart shown in FIG. 7 for the time series endoscopic images Ia constituting the video image. Then, the image processing device 1 terminates the process of the flowchart upon determining that the target video image has ended at step S18. In contrast, it gets back to the process at step S11 upon determining that the target video image has not ended, and proceeds with the process of the flowchart for the subsequent endoscopic image Ia in the time series.

### (Second Modification)

The adequacy determination unit 32 may make the progression adequacy determination on the basis of the degree of reliability calculated separately from the degree of reliability outputted by the lesion detection model.

In this case, for example, if the lesion detection unit 31 detects a lesion region, the adequacy determination unit 32 calculates the degree of reliability based on the endoscopic image Ia including the detected lesion region. In this case, for example, the adequacy determination unit 32 calculates the degree of reliability from the endoscopic image Ia by using a model configured to output, when an endoscopic image is inputted thereto, a reliability score of the presence of a lesion region in the inputted endoscopic image. In this case, for example, the above-described model is a model based on machine learning such as a neural network, and learned parameters are stored in advance in the memory 12 or the like. The above-described model may be a classification model that conducts binary classification as to the presence or absence of a lesion region. In this case, the adequacy determination unit 32 acquires, as the above-mentioned degree of reliability, the confidence score corresponding to the class of the presence of the lesion region outputted by the above-mentioned classification model.

According to this modification, the adequacy determination unit 32 can suitably make the progression adequacy determination.

### (Third Modification)

The lesion detection model information D1 and the progression determination model information D2 may be stored in a storage device separate from the image processing device 1.

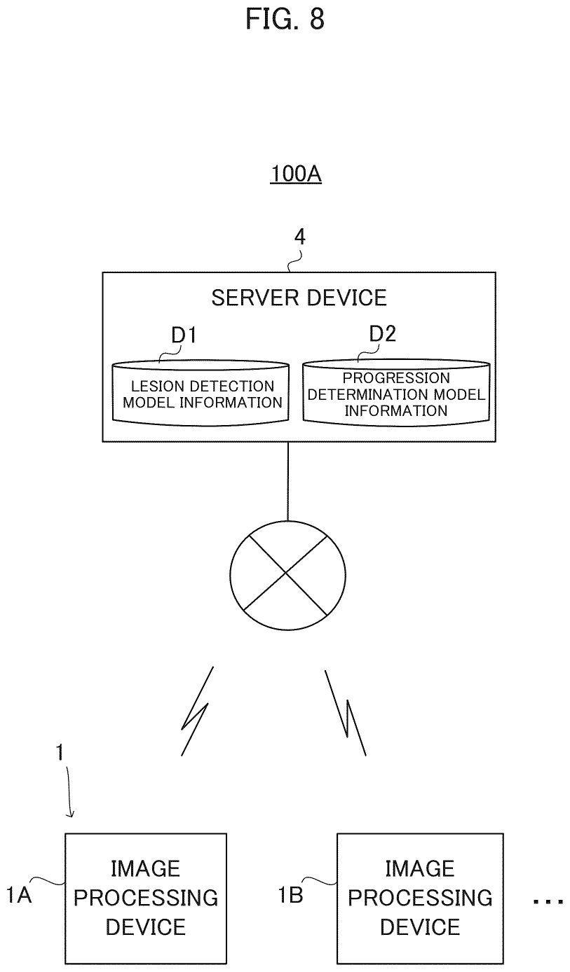

FIG. 8 is a schematic configuration diagram of an endoscopic examination system 100A according to the modification. For simplicity, the display device 2 and the endoscope 3 and the like are not shown herein. The endoscopic examination system 100A includes a server device 4 that stores the lesion detection model information D1 and the progression determination model information D2. Further, the endoscopic examination system 100A includes a plurality of image-processing devices 1 (1A, 1B, . . . ) capable of data communication with the server device 4 via a network.

In this instance, each image processing device 1 refers to the lesion detection model information D1 and the progression determination model information D2 through the network. In this case, the interface 13 of each image processing device 1 includes a communication interface such as a network adapter for data communication. According to this configuration, each image processing device 1 refers to the lesion detection model information D1 and the progression determination model information D2 as in the above-described example embodiment to suitably execute the process relating to the determination of the degree of progression of the lesion. The server device 4 may execute at least a part of the process to be performed by each function block of the processor 11 of the image processing device 1 illustrated in FIG. 4 instead.

### Second Example Embodiment

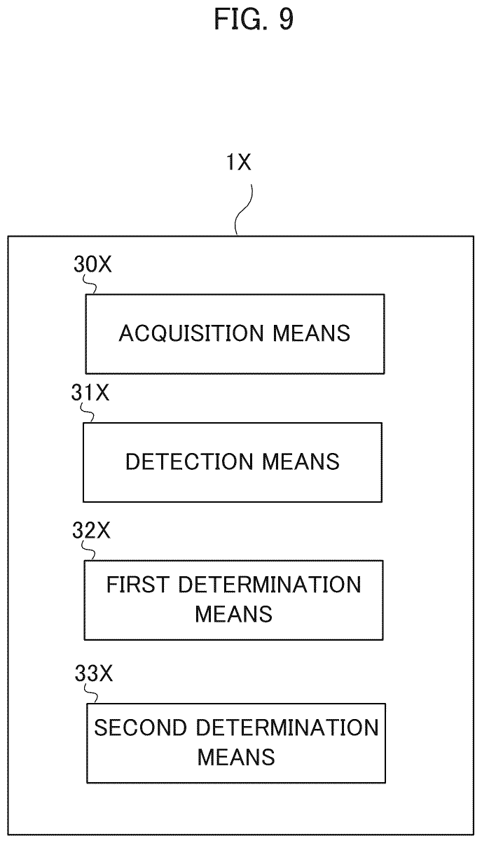

FIG. 9 is a block diagram of the image processing device 1X according to the second example embodiment. The image processing device 1X includes an acquisition means 30X, a detection means 31X, a first determination means 32X, and a second determination means 33X. The image processing device 1X may be configured by a plurality of devices.

The acquisition means 30X is configured to acquire an endoscopic image obtained by photographing an examination target. Examples of the acquisition means 30X include the endoscopic image acquisition unit 30 in the first example embodiment (including modifications, hereinafter the same). The acquisition means 30X may be configured to immediately acquire the endoscopic image generated by the photographing unit, or may acquire, at a predetermined timing, the endoscopic image stored in the storage device generated by the photographing unit in advance.

The detection means 31X is configured to detect, based on the endoscopic image, a lesion region which is a candidate region of a lesion of the examination target in the endoscopic image. Examples of the detection means 31X include the lesion detection unit 31 in the first example embodiment.

The first determination means 32X is configured to determine whether or not the endoscopic image is an image adequate to determine a degree of progression or an invasion depth, based on at least one of a size of the lesion region and/or a degree of reliability regarding a probability of the lesion region as the lesion. Examples of the first determination means 32X include the adequacy determination unit 32 in the first example embodiment.

The second determination means 33X is configured to determine the degree of progression or the invasion depth, based on the endoscopic image determined to be the image adequate to determine the degree of progression or the invasion depth. Examples of the second determination means 33X include the progression determination unit 33 in the first example embodiment.

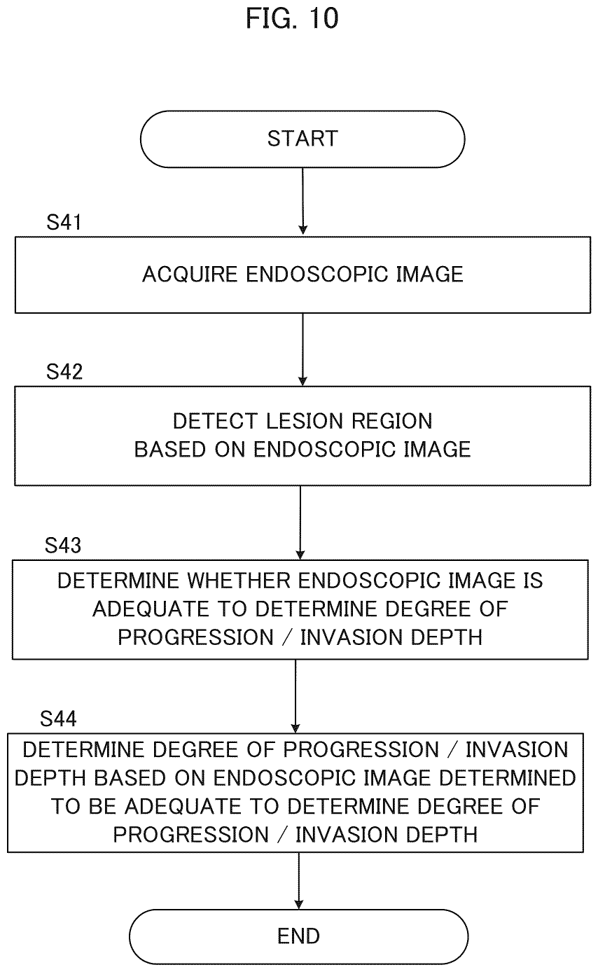

FIG. 10 is an example of a flowchart showing a processing procedure in the second example embodiment. The acquisition means 30X acquires an endoscopic image obtained by photographing an examination target (step S21). The detection means 31X detects, based on the endoscopic image, a lesion region which is a candidate region of a lesion of the examination target in the endoscopic image (step S22). The first determination means 32X determines whether or not the endoscopic image is an image adequate to determine a degree of progression or an invasion depth, based on at least one of a size of the lesion region and/or a degree of reliability regarding a probability of the lesion region as the lesion (step S23). The second determination means 33X determines the degree of progression or the invasion depth, based on the endoscopic image determined to be the image adequate to determine the degree of progression or the invasion depth (step S24).

According to the second example embodiment, the image processing device 1X can accurately determine the degree of progression or the invasion depth based on the selected endoscopic image.

In the example embodiments described above, the program is stored by any type of a non-transitory computer-readable medium (non-transitory computer readable medium) and can be supplied to a control unit or the like that is a computer. The non-transitory computer-readable medium include any type of a tangible storage medium. Examples of the non-transitory computer readable medium include a magnetic storage medium (e.g., a flexible disk, a magnetic tape, a hard disk drive), a magnetic-optical storage medium (e.g., a magnetic optical disk), CD-ROM (Read Only Memory), CD-R, CD-R/W, a solid-state memory (e.g., a mask ROM, a PROM (Programmable ROM), an EPROM (Erasable PROM), a flash ROM, a RAM (Random Access Memory)). The program may also be provided to the computer by any type of a transitory computer readable medium. Examples of the transitory computer readable medium include an electrical signal, an optical signal, and an electromagnetic wave. The transitory computer readable medium can provide the program to the computer through a wired channel such as wires and optical fibers or a wireless channel.

The whole or a part of the example embodiments described above (including modifications, the same applies hereinafter) can be described as, but not limited to, the following Supplementary Notes.

### [Supplementary Note 1]

An image processing device comprising:
an acquisition means configured to acquire an endoscopic image obtained by photographing an examination target;
a detection means configured to detect, based on the endoscopic image, a lesion region which is a candidate region of a lesion of the examination target in the endoscopic image;
a first determination means configured to determine whether or not the endoscopic image is an image adequate to determine a degree of progression or an invasion depth, based on at least one of a size of the lesion region and/or a degree of reliability regarding a probability of the lesion region as the lesion; and
a second determination means configured to determine the degree of progression or the invasion depth, based on the endoscopic image determined to be the image adequate to determine the degree of progression or the invasion depth.

### [Supplementary Note 2]

The image processing device according to Supplementary Note 1,
wherein the first determination means is configured to change a criterion to be used for determining whether or not the endoscopic image is an image adequate to determine the degree of progression or the invasion depth, based on information obtained by determining the degree of progression or the invasion depth.

### [Supplementary Note 3]

The image processing device according to Supplementary Note 2,
wherein the first determination means is configured to change the criterion, based on a degree of confidence for a class, determined by the second determination means, of the degree of progression or the invasion depth.

### [Supplementary Note 4]

The image processing device according to Supplementary Note 3,
wherein the first determination means is configured to change the criterion, based on a degree of change in the class, determined in time series by the second determination means, of the degree of progression or the invasion depth.

### [Supplementary Note 5]

The image processing device according to Supplementary Note 3,
wherein the criterion is at least one of a first criterion regarding the size of the lesion region and/or a second criterion regarding the degree of reliability.

### [Supplementary Note 6]

The image processing device according to Supplementary Note 1, further comprising
an output control means configured to output, by a display device or audio output device, a suggestion regarding photography of the endoscopic image, upon determining that the endoscopic image is not the image adequate to determine the degree of progression or the invasion depth.

### [Supplementary Note 7]

The image processing device according to Supplementary Note 6,
wherein the output control means is configured to output information indicating a target range of the lesion region on the endoscope image.

### [Supplementary Note 8]

The image processing device according to Supplementary Note 7,
wherein the output control means is configured to determine at least one of a shape of the target range and/or a size of the target range, based on a detection result of the lesion region by the detection means.

### [Supplementary Note 9]

The image processing device according to Supplementary Note 6,
wherein the output control means is configured to output information prompting a photographing position to approach the lesion region as the suggestion.

### [Supplementary Note 10]

The image processing device according to Supplementary Note 1,
wherein the detection means is configured to acquire an inference result regarding the lesion region outputted from a lesion detection model by inputting the endoscopic image to the lesion detection model, and
wherein the lesion detection model is a model obtained by machine learning of a relation between an input image to the lesion detection model and the lesion region included in the input image.

### [Supplementary Note 11]

The image processing device according to Supplementary Note 6,
wherein the output control means is configured to output the suggestion to assist examiner's decision making.

### [Supplementary Note 12]

An image processing method executed by a computer, the image processing method comprising:
acquiring an endoscopic image obtained by photographing an examination target;
detecting, based on the endoscopic image, a lesion region which is a candidate region of a lesion of the examination target in the endoscopic image;
determining whether or not the endoscopic image is an image adequate to determine a degree of progression or an invasion depth, based on at least one of a size of the lesion region and/or a degree of reliability regarding a probability of the lesion region as the lesion; and
determining the degree of progression or the invasion depth, based on the endoscopic image determined to be the image adequate to determine the degree of progression or the invasion depth.

### [Supplementary Note 13]

A storage medium storing a program executed by a computer, the program causing the computer to:
acquire an endoscopic image obtained by photographing an examination target;
detect, based on the endoscopic image, a lesion region which is a candidate region of a lesion of the examination target in the endoscopic image;
determine whether or not the endoscopic image is an image adequate to determine a degree of progression or an invasion depth, based on at least one of a size of the lesion region and/or a degree of reliability regarding a probability of the lesion region as the lesion; and
determine the degree of progression or the invasion depth, based on the endoscopic image determined to be the image adequate to determine the degree of progression or the invasion depth.

While the invention has been particularly shown and described with reference to example embodiments thereof, the invention is not limited to these example embodiments. It will be understood by those of ordinary skilled in the art that various changes in form and details may be made therein without departing from the spirit and scope of the present invention as defined by the claims. In other words, it is needless to say that the present invention includes various modifications that could be made by a person skilled in the art according to the entire disclosure including the scope of the claims, and the technical philosophy. All patent and Non-patent Literatures mentioned in this specification are incorporated by reference in its entirety.

### DESCRIPTION OF REFERENCE NUMERALS

1, 1A, 1B, 1X Image processing device
2 Display device
3 Endoscope
11 Processor
12 Memory
13 Interface
14 Input unit
15 Light source unit
16 Audio output unit
100, 100A Endoscopic examination system

What is claimed is:

1. An image processing device comprising:
at least one memory configured to store instructions; and
at least one processor configured to execute the instructions to:
acquire an endoscopic image obtained by photographing an examination target;
detect a lesion region which is a candidate region of a lesion of the examination target in the endoscopic image by inputting the endoscopic image to a lesion detection model trained a relationship between an input image and the lesion region included in the input image by machine learning;
determine whether or not the endoscopic image is an image adequate to determine a degree of progression or an invasion depth, based on at least one of a size of the lesion region by inputting the endoscopic image to a progression determination model trained a relationship between an input image and the degree of progression of the lesion in the input image by machine learning; and
determine the degree of progression or the invasion depth, based on the endoscopic image determined to be the image adequate to determine the degree of progression or the invasion depth.

2. The image processing device according to claim 1,
wherein the at least one processor is configured to execute the instructions to change a criterion to be used for determining whether or not the endoscopic image is an image adequate to determine the degree of progression or the invasion depth, based on information obtained by determining the degree of progression or the invasion depth.

3. The image processing device according to claim 2,
wherein the at least one processor is configured to execute the instructions to change the criterion, based on a degree of confidence for a class of the degree of progression or the invasion depth.

4. The image processing device according to claim 3,
wherein the at least one processor is configured to execute the instructions to change the criterion, based on a degree of change in the class, determined in time series, of the degree of progression or the invasion depth.

5. The image processing device according to claim 3,
wherein the criterion is at least one of a first criterion regarding the size of the lesion region and/or a second criterion regarding the degree of reliability.

6. The image processing device according to claim 1,
wherein the at least one processor is configured to further execute the instructions to output, by a display device or audio output device, a suggestion regarding photography of the endoscopic image, upon determining that the endoscopic image is not the image adequate to determine the degree of progression or the invasion depth.

7. The image processing device according to claim 6,
wherein the at least one processor is configured to execute the instructions to output information indicating a target range of the lesion region on the endoscope image.

8. The image processing device according to claim 7,
wherein the at least one processor is configured to execute the instructions to determine at least one of a shape of the target range and/or a size of the target range, based on a detection result of the lesion region.

9. The image processing device according to claim 6,
wherein the at least one processor is configured to execute the instructions to output information prompting a photographing position to approach the lesion region as the suggestion.

10. The image processing device according to claim 1,
wherein the lesion detection model is trained the relationship between the input image and, at least one of a lesion reliability map relating to the lesion included in the input image or a bounding box surrounding the lesion included in the input image by machine learning.

11. The image processing device according to claim 6,
wherein the at least one processor is configured to execute the instructions to output the suggestion to assist examiner's decision making.

12. An image processing method executed by a computer, the image processing method comprising:
acquiring an endoscopic image obtained by photographing an examination target;
detecting a lesion region which is a candidate region of a lesion of the examination target in the endoscopic image by inputting the endoscopic image to a lesion detection model trained a relationship between an input image and the lesion region included in the input image by machine learning;
determining whether or not the endoscopic image is an image adequate to determine a degree of progression or an invasion depth, based on at least one of a size of the lesion region by inputting the endoscopic image to a progression determination model trained a relationship between an input image and the degree of progression of the lesion in the input image by machine learning; and
determining the degree of progression or the invasion depth, based on the endoscopic image determined to be the image adequate to determine the degree of progression or the invasion depth.

13. The image processing method according to claim 12, further comprising,
changing a criterion to be used for determining whether or not the endoscopic image is an image adequate to determine the degree of progression or the invasion depth, based on information obtained by determining the degree of progression or the invasion depth.

14. The image processing method according to claim 13, further comprising,
changing the criterion, based on a degree of confidence for a class of the degree of progression or the invasion depth.

15. The image processing method according to claim 14, further comprising,
changing the criterion, based on a degree of change in the class, determined in time series, of the degree of progression or the invasion depth.

16. The image processing method according to claim 14,
wherein the criterion is at least one of a first criterion regarding the size of the lesion region and/or a second criterion regarding the degree of reliability.

17. The image processing method according to claim 12, further comprising,
outputting, by a display device or audio output device, a suggestion regarding photography of the endoscopic image, upon determining that the endoscopic image is not the image adequate to determine the degree of progression or the invasion depth.

18. The image processing method according to claim 17, further comprising,
outputting information indicating a target range of the lesion region on the endoscope image.

19. The image processing method according to claim 18, further comprising,
determining at least one of a shape of the target range and/or a size of the target range, based on a detection result of the lesion region.

20. A non-transitory computer readable storage medium storing a program executed by a computer, the program causing the computer to:
acquire an endoscopic image obtained by photographing an examination target;
detect a lesion region which is a candidate region of a lesion of the examination target in the endoscopic image by inputting the endoscopic image to a lesion detection model trained a relationship between an input image and the lesion region included in the input image by machine learning;
determine whether or not the endoscopic image is an image adequate to determine a degree of progression or an invasion depth, based on at least one of a size of the lesion region by inputting the endoscopic image to a progression determination model trained a relationship between an input image and the degree of progression of the lesion in the input image by machine learning; and
determine the degree of progression or the invasion depth, based on the endoscopic image determined to be the image adequate to determine the degree of progression or the invasion depth.
